(19) United States
(12) Patent Application Publication
(10) Pub. No.: US 2024/0041383 A1
(43) Pub. Date: Feb. 8, 2024

(54) DEVICES, SYSTEMS, AND METHODS FOR QUANTIFYING NEURO-INFLAMMATION

(71) Applicant: Gen Shinozaki, Iowa City, IA (US)

(72) Inventor: Gen Shinozaki, Iowa City, IA (US)

(21) Appl. No.: 18/266,128

(22) PCT Filed: Dec. 13, 2021
(86) PCT No.: PCT/US21/63166

§ 371 Date: Jun. 8, 2023

Related U.S. Application Data
(60) Provisional application No. 63/124,524, filed on Dec. 11, 2020.

(51) Int. Cl. A61B 5/384; A61B 5/374

(52) U.S. Cl. CPC A61B 5/384; A61B 5/374

(57) ABSTRACT

Disclosed and contemplated herein is a bispectral EEG (BSEEG) method which can detect patients with delirium and can detect delirium with systemic inflammation. In various implementations, a mouse model is used to detect delirium with systemic inflammation induced by lipopolysaccharide (LPS) injection.

### CROSS-REFERENCE TO RELATED APPLICATION(S)

This application claims the benefit under 35 U.S.C. § 119(e) to U.S. Provisional Application 63/124,524, filed Dec. 11, 2020, and entitled “Devices, Systems, and Method for Quantifying Neuro-Inflammation,” which is hereby incorporated herein by reference in its entirety for all purposes.

### TECHNICAL FIELD

The disclosure relates to quantifying neuro-inflammation and in particular to uses of bispectral EEG (BSEEG) for quantifying neuro-inflammation.

### BACKGROUND

It is widely recognized that delirium is not only common but dangerous, with one-year mortality as high as 40%. Delirium has, moreover, been associated with extended length of stay in the hospital, decreased chance of discharge to home, and long-term cognitive impairment. Further, delirium comes with significant financial burden: it is estimated that one case of delirium can cost —$60,000. With 2-3 million cases of delirium per year in the U.S. alone, the annual financial loss associated with delirium is —$150 billion. Delirium thus represents a significant burden for elderly patients, healthcare providers, hospital systems, and the healthcare economy.

Despite the burden delirium presents, little is known of its pathophysiology, and no effective preventative measures or treatments have been identified. Moreover, diagnosis of delirium relies largely on subjective assessment of mental status based on a psychiatric interview.

Thus, there is a need in the art for devices, systems, and methods to systematically dissect variables relevant to delirium including neuro-inflammation.

### BRIEF SUMMARY

Disclosed herein are various methods for detecting and quantifying neuro-inflammation and associated delirium in patients.

In the various Examples described herein, a system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. Various implementations include corresponding computer systems, apparatus, and computer programs recorded on one or more computer storage devices, each configured to perform the actions of the methods.

In Example 1, a bispecteral EEG assessment system, comprising at least one sensor, a screening device, and a platform tool, the system being configured to record an EEG to obtain raw EEG data, and calculate a recorded BSEEG score, and compare the recorded BSEEG score to a clinical inflammation threshold.

In Example 2, the system of Example 1, wherein the raw EEG data is recorded over a time period.

In Example 3, the system of any of Examples 1-2, further comprising calculating a maximum BSEEG score.

In Example 4, the system of any of Examples 1-3, further comprising outputting the recorded BSEEG score.

In Example 5, the system of any of Examples 1-4, wherein the system is configured to update the clinical inflammation threshold via a machine learning model.

In Example 6, the system of any of Examples 1-5, wherein the platform tool is a web-based tool.

In Example 7, the system of any of Examples 1-6, further configured to quantify the level of brain wave abnormality and the level of neuroinflammation over time.

In Example 8, the system of any of Examples 1-7, wherein the recorded BSEEG score is calculated from raw EEG data via power spectral density analysis.

In Example 9, the system of any of Examples 1-8, where the recorded BSEEG score is a ratio of EEG signals at 3 Hz to 10 Hz.

In Example 10, the system of any of Examples 1-9, further comprising calculating a baseline BSEEG score.

In Example 11, the system of any of Examples 1-10, further comprising calculating a standardized BSEEG score.

In Example 12, a system for quantifying neuroinflammation, comprising: at least two sensors configured to record EEG signals indicative of brain wave frequencies; a processor; and at least one tool configured to run on the processor, the tool configured to record EEG signals, perform spectral density analysis on the EEG signal to calculate a BSEEG score, and compare the BSEEG score to a clinical inflammation threshold to determine an amount of neuroinflammation.

In Example 13, the system of any of Example 12, wherein the at least one tool is configured to determine a baseline BSEEG score.

In Example 14, the system of any of Examples 12-13, wherein the at least one tool is configured to calculate a standardized BSEEG score.

In Example 15, the system of any of Examples 12-14, wherein the at least one tool is configured to calculate a maximum BSEEG score over a given time period.

In Example 16, the system of any of Examples 12-15, wherein the at least one tool is configured to calculate an average BSEEG score over a given time period.

In Example 17, the system of any of Examples 12-16, further comprising a display configured to display output including an average BSEEG score, a maximum BSEEG score, a baseline BSEEG score, a standardized BSEEG score.

In Example 18, the system of any of Examples 12-17, wherein the tool is further configured to quantify a level of brain wave abnormality and a level of neuroinflammation over time.

In Example 19, the system of any of Examples 12-18, wherein the given time period is about 12 hours.

In Example 20, an inflammation assessment method, comprising: recording an EEG to obtain raw EEG data; calculating a recorded BSEEG score; and comparing the recorded BSEEG score to a clinical inflammation threshold.

Implementations of the described techniques may include hardware, a method or process, or computer software on a computer-accessible medium. While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is a system diagram, according to one implementation.

FIG. 2A is a flow diagram of the system, according to one implementation.

FIG. 2B is a flow diagram for various system calculations, according to one implementation.

FIG. 3 shows an experimental schedule, including EEG head mount placement, baseline EEG measurement, followed by lipopolysaccharide (LPS) injection to induce systemic inflammation to assess brain response measured by EEG change.

FIG. 4 shows a diagram of electrode placement on mouse head. A total of 4 electrodes (2 EEG, a ground, and a reference) were placed following standard procedures.

FIG. 5 shows a typical pattern of BSEEG scores in mice after LPS injection. EEG 1 and EEG 2 are recorded from a head mount located as shown in FIG. 2 and independent recordings from the same mouse in the same experiment were taken. The BSEEG scores from both channels increased after injection with LPS. The BSEEG score remained abnormally elevated for several days before returning to baseline. Also, diurnal changes seen prior to LPS administration diminished during the period of BSEEG elevation, consistent with the clinical picture of patients with delirium, who often experience abnormal sleep cycles. The example shown here is of a young mouse injected with 1 mg/kg LPS.

FIG. 6 shows the diminished diurnal change of standardized BSEEG (sBSEEG) score after various dosages of LPS injection. The average sBSEEG score from young mice-EEG2 (frontal) increased after injection with LPS. The score remained abnormally elevated for several days before returning to baseline. Also, diurnal changes seen prior to LPS administration diminished during the period of sBSEEG elevation, consistent with the clinical picture of patients with delirium, who often experience abnormal sleep cycles.

FIG. 7 shows dose-dependent changes in BSEEG score in mice after LPS injection. Maximum BSEEG scores after various doses of LPS injection are compared. Dose-dependent increases in BSEEG scores after LPS injection were shown. Error bars are S.E.M.

FIG. 8 shows an experimental schedule, including EEG head mount surgery, EEG recording, and UP-LPS injection.

FIG. 9 shows a typical BSEEG result in a young mouse before and after a saline injection.

FIG. 10 shows sBSEEG results for a young mouse before and after a saline injection.

FIG. 11 shows a comparison of BSEEG and sBSEEG results in young mice before and after a UP-LPS injection.

FIG. 12 shows a dose dependent increase in sBSEEG scores after UP-LPS injection in young mice.

FIG. 13A shows a dose dependent increase in sBSEEG scores after UP-LPS injection in young mice.

FIG. 13B shows a dose dependent increase in sBSEEG scores after UP-LPS injection in aged mice.

FIG. 14 shows a comparison of the sBSEEG score increase of both young mice and aged mice after a 2.0 mg/kg injection of UP-LPS.

### DETAILED DESCRIPTION

Disclosed herein are various methods and related systems and devices for use of a bispectral EEG (BSEEG) with an algorithm to evaluate, study, and quantify cognitive disturbances such as delirium, neuro-inflammation, and the like. In certain implementations, the method and system may be used to predict mortality. In various implementations, the disclosed BSEEG method can objectively quantify the level of neuro-inflammation induced by systemic inflammation via lipopolysaccharides (LPS).

Disclosed and contemplated herein is a further bispectral EEG (BSEEG) approach. In certain aspects, the disclosed systems, devices, and methods can utilize the detection of patients with delirium and determine the association with systemic inflammation. In certain further aspects, the disclosed methods, systems, and devices provide a more objective and efficient approach than commonly used behavioral tests or pathological examinations for detection of delirium. Further, in certain implementations, a web-based tool for BSEEG and sBSEEG scoring may be used in conjunction with the method, as will be discussed further herein.

Without desiring to be bound to any particular mechanism, it has been suggested that neuroinflammation plays a key role in the pathophysiology of delirium. Evidence suggests that microglia, which are the resident immune cells of the central nervous system and contribute to neuroinflammation, are easily activated and release inflammatory cytokines induced by peripheral infection. A number of animal studies have shown that aged rodents treated with LPS show activated microglia and increased protein levels of inflammatory cytokines compared to adult rodents. Although these molecular and pathological changes were observed with cognitive deficit-like behavioral change, few studies show a relationship between microglial activation and EEG change. Because the method and systems discussed herein can quantify the EEG slowing by calculating a BSEEG score, the disclosed method can make it possible to more accurately assess correlations between microglial activation and brain electrophysiological dysfunction induced by LPS or other types of insult.

Previous animal studies using inflammation-induced cognitive change have relied on behavioral testing without objective and biological methods to quantify the severity of cognitive disturbances. As shown and described herein, a mouse model is used to detect delirium with systemic inflammation induced by lipopolysaccharide (LPS) injection. BSEEG data was used to objectively quantify brain electrophysiological changes in response to LPS injection inducing a systemic inflammation reaction in C57Bl/6 mice.

A previously disclosed approach for detecting delirium utilized bispectral EEG (BSEEG) and an algorithm in clinical study. This prior known approach effectively differentiates between patients with and without delirium, and in certain implementations predicts long-term mortality.

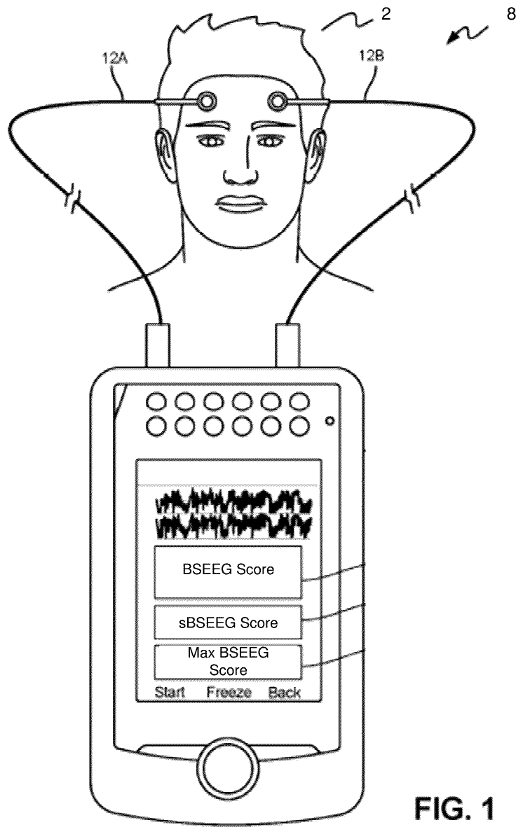

In various implementations, the disclosed system and method include a screening device 8, shown for example in FIG. 1. In these implementations, the screening device 8 is configured to receive signals from one or more sensors 12A, 12B. In this implementation, the one or more sensors 12A, 12B are brain sensors, such as, but not limited to, electrodes placed on a patient 2. The one or more sensors 12A, 12B are configured to measure brain activity of the patient 2, such as via electroencephalography (EEG). The signals may then be processed to extract one or more features of the signals. The Sensors 12A, 12B may be wired or wireless. The one or more features may be analyzed to determine one or more values for each of the one or more features. The values may include a BSEEG score, a standardized BSEEG score (sBSEEG score), and a maximum BSEEG score. The values may be compared to various data to determine the severity of neuroinflammation and delirium.

In certain implementations, the screening device 8 and/or disclosed system and methods include one or more computers configured to perform particular operations or actions by virtue of having software, firmware, hardware, or a combination of them install on the system/device 8 that in operation causes or cause the system/device 8 to perform the actions. One or more computer programs can be configured to perform particular operations or actions by virtue of including instructions that, when executed by data processing apparatus, cause the apparatus to perform actions.

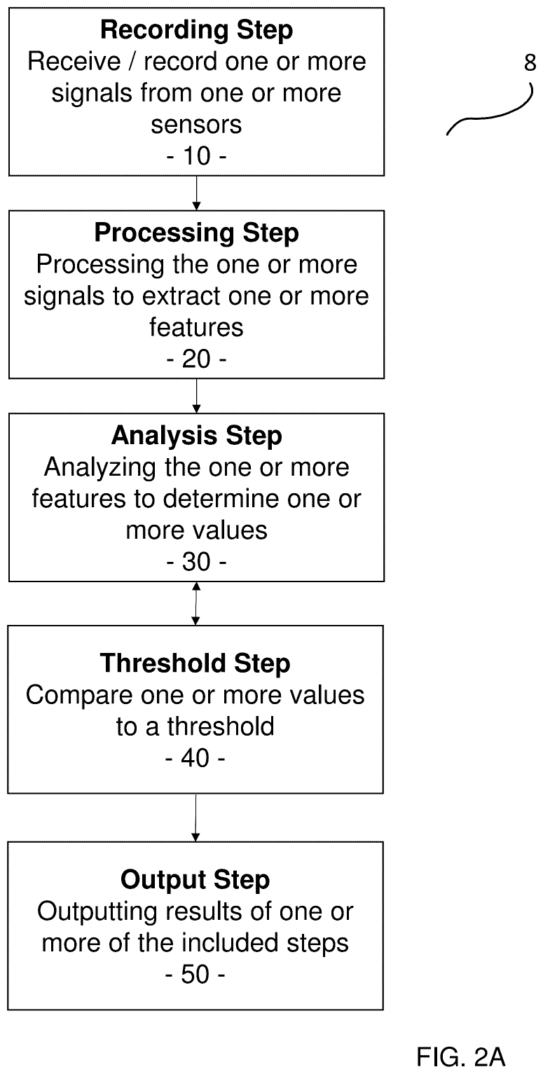

Turning now to FIGS. 2A and 2B, the approach disclosed herein includes a series of steps, each of which is optional and certain steps may be performed in any order and may be conducted iteratively. In one step, an EEG is recorded (box 10), to obtain raw EEG data (box 12). In a further processing step (box 20), the EEG recordings are converted via power spectral density (box 22) to calculate a BSEEG score (box 24). In certain implementations, the BSEEG score (box 24) is calculated using a similar BSEEG algorithm disclosed in PCT Application No. PCT/US16/64937, filed Dec. 5, 2017, and entitled “Apparatus, Systems and Methods for Predicting, Screening and Monitoring of Encephalopathy Delirium,” PCT Application No. PCT/US19/51276, filed Sep. 16, 2019, and entitled “Systems and Methods for Detection of Delirium Risk Using Epigenetic Markers” and PCT/US20/26914, filed Apr. 6, 2020, and entitled “Apparatus, Systems and Methods for Predicting, Screening and Monitoring of Mortality and Other Conditions,” each of which is incorporated herein by reference.

That is, in one step, after recording raw EEG signals (box 12), the data can be exported, such as in an EDF file by a default software for the Pinnacle Sirenia EEG system or other appropriate file type and system, as would be readily appreciated by those of skill in the art. The exported file may be uploaded to a platform tool, such as a web-based tool, or other software application, and the BSEEG score (box 24) can be visualized and the results viewed, as would be readily appreciated.

In another optional step, once sequential data from recording over days is uploaded, an sBSEEG score (box 26) can be calculated using the baseline data. The sBSEEG score being calculated as the change in BSEEG score from a baseline measurement (box 28).

In a further analysis step (box 30), an average BSEEG score (box 32) can be calculated over a given time period, such as over a 12-hour period. This average BSEEG score (box 32) can then be used to calculate a maximum BSEEG score (box 34) for a discrete period within the period from which the average was taken. For example, the maximum BSEEG score may be equal to a comparison of a BSEEG score for each hour in the 12-hour period the average BSEEG score was calculated and the average BSEEG score. The highest value after comparison of each time period is the maximum BSEEG score (box 34).

In certain implementations, in an optional step, the recorded BSEEG score and/or sBSEEG score is compared to a clinical threshold, such as a clinical inflammation threshold (box 40), wherein results that exceed the clinical inflammation threshold are reported for clinical use, such as via the platform tool.

In certain implementations the threshold step (box 40) includes determining an inflammation threshold. In these and other implementations, the inflammation threshold may be based on historical EEG data for the patient or a group of patients, medical record data, and additional data sources as would be appreciated.

In a further optional step, the recorded BSEEG score, sBSEEG score, and/or threshold comparison are processed and analyzed by the system 10 is outputted in an output step (box 50). The outputted data can be read and interpreted by a physician, patient, or other stakeholder. High sBSEEG and BSEEG scores are indicative of a higher level of neuroinflammation and beyond a specified threshold indicate abnormal brain activity. In various implementations, the outputted information is displayed on the device 8, as shown for example in FIG. 1.

In various implementations, the disclosed methods, systems, and devices allow for the improved assessment of delirium using electroencephalography EEG, which can capture diffuse slow waves, such as delta waves, that are characteristic of delirium in humans. A bispectral EEG method using an algorithm was previously disclosed in the incorporated references that, by processing brain waves using spectral power density analysis and capturing slow waves compared to high-frequency waves, effectively differentiates between patients with and without delirium. A human study of the BSEEG method in elderly patients admitted to the University of Iowa Hospitals and Clinics revealed sensitivity and specificity of over 80%, with a receiver operating characteristic (ROC) of ˜0.8. This result suggested that the BSEEG score can be a biomarker of delirium.

Many prior EEG studies have used anticholinergic agents such as atropine or biperiden to induce delirium features of EEG in animal models. Yet, this scenario is relevant only to rare cases of overdoses of anticholinergic medications. Disclosed herein is a model of delirium in which lipopolysaccharide (LPS) is injected to model systemic inflammation induced delirium. Further, the disclosed quantification methods are relevant to clinical contexts and directly translatable to clinical settings to understand the pathophysiology of delirium in humans, and specifically the effect of peripheral inflammation/infection leading to inflammation in the central nervous system.

While delirious patients are recovering from systemic inflammation such as pneumonia or urosepsis. The data disclosed herein shows that use of BSEEG in conjunction with the algorithm makes it possible to objectively and precisely quantify electrophysiological abnormality, or delirium.

Further disclosed herein is an EEG-based technology capable of capturing electrophysiological features characteristic of systemic inflammation-related delirium. A web-based tool may be used to create wide reaching applicability of the disclosed method and system. In certain implementations, users can upload their own EEG recordings to visualize BSEEG score changes over time. In various implementations, the web-based tool can quantify the level of brain wave abnormality and the level of neuroinflammation over time.

As described in the examples herein, BSEEG was used in mice to detect electrophysiological changes consistent with delirium in humans. Further it is shown that aged mice are more sensitive to LPS than young mice, as reflected by elevated BSEEG score. To evaluate the dose-dependent EEG change from LPS injection, BSEEG response to a range of LPS concentrations was evaluated. These age-associated and LPS dose-associated differences are consistent with the fact that patients with higher age or with severer systemic inflammation, such as serious infection or highly invasive surgery, are at higher risk of developing delirium.

As delirium can be caused by multifactorial etiology including infection, surgery, cholinergic medication overdose, and more, it is ideal to compare different triggering methods to compare BSEEG change along with various etiology. Further, it is possible that the inhibition or activation of microglia may alter BSEEG score change in response to LPS or other triggers. Certain medication may suppress occurrence of delirium can also prevent BSEEG score from increasing in response to LPS, such as ramelteon, suvorexant, and dexamethasone.

In further implementations, the relationship between BSEEG scores and behavioral change can be evaluated, in these methods a wireless EEG system may be used allowing for measurement of both EEG and behavioral response at the same time from the same patient. To evaluate the consistency of EEG change and behavioral change, such an approach would provide valuable information. That is, observed behavioral changes can be compared/correlated with BSEEG and sBSEEG scores. In addition, to better assess the usefulness of BSEEG in evaluation of brain dysfunction or delirium, it is important to assess correlations between BSEEG score and pathological data such as microglia activation, as well as molecular data such as cytokine levels and gene expression. In various implementations such correlations allow for understanding of the pathophysiological mechanism of brain function abnormality and neuroinflammation.

In certain implementations a machine learning model is used to identify characteristics of the BSEEG/sBSEEG scores and establish parameters and thresholds, and can be used to revise the other systems, methods, and devices described herein, such as by refining the thresholds and standards to improve the accuracy of the system and device 8. In these implementations, a model is used to associate digital BSEEG data within a computing machine, such as a server or database.

Generally, the various machine learning approaches, may be coded for execution on the processor, server/computing device, a database, third party server other computing or electronic storage device in operable communication with the device 8 and/or sensors 12A, 12B.

The model may be executed on BSEEG data recorded or otherwise observed from patients, such as but not limited to patient movement recorded via a wearable device or motion capture through video imaging, as well as from other recorded data, such as from medical records.

In various implementations, the data may include, but is not limited to, one or more of the following BSEEG data, raw EEG data, power spectral density converted data, and other implementations as would be appreciated.

Accordingly, the various systems and methods using the machine learning model may send and/or receive information from various computing devices, as well as a patient's electronic medical records (“EMR”) for use in monitoring, screening, or predicting of delirium/inflammation by way of a gateway or other connection mechanism. In certain embodiments, the systems and methods may utilize EMR data to improve accuracy of the monitoring, screening, or predicting of delirium performed in conjunction with the screening device 8 and associated system and methods.

In various implementations, BSEEG scores may also be loaded on to any of the computer storage devices of a computer to generate an appropriate tree algorithm or logistic regression formula. Once generated, the tree algorithm, which may take the form of a large set of if-then conditions, may then be coded using any general computing language for test implementation. For example, the if-then conditions can be captured and compiled to produce a machine-executable module, which, when run, accepts new data and outputs results, which can include a calculated prediction or other graphical representation.

The output may be in the form of a graph indicating the prediction or probability value along with related statistical indicators such as p-values, chi-scores and the like. In various implementations, these results can be re-introduced into the learning module or elsewhere to continually improve the functions of the system, including by updating the various thresholds used throughout. It is understood that these implementations are also able to trend the respective data values and readings to improve the performance of the device, system and methods. In these implementations, for example, a continuous stream of trend data that can be used to provide additional optional evaluation steps, and trends over time can be identified. In various implementations, the model can provide additional program to improve accuracy, as well as be included in aggregation and adjustment of the various thresholds described herein.

As used herein, the terms “value” and “features” can be interchangeable, and contemplate raw and analyzed data, be it numerical, time-scale, graphical, or other. In various implementations like those described herein, the value, such as the number of high frequency waves may be compared against a threshold. Alternatively, or in addition, a ratio of two or more values may be compared against a threshold. The threshold may be a predetermined value. The threshold may be based on statistical information regarding the presence, absence, or likelihood of subsequent development of delirium, such as information from a population of individuals. In certain embodiments, the threshold may be predetermined for one or more patients. In certain embodiments, the threshold may be consistent for all patients. In certain embodiments, the threshold may be specific to one or more characteristics of the patient, such as current health, age, gender, race, medical history, other medical conditions, and the like. In certain embodiments, the threshold may be adjusted based on physiological data in a patient's EMR.

In certain embodiments, the threshold may be a ratio of high frequency waves to low frequency waves. In certain embodiments, the threshold may be a ratio of high frequency waves over a period of time to low frequency waves over a period of time. Throughout the disclosure herein, the ratio is referred to as the ratio of high frequency waves to low frequency waves, but it is understood that the ratio could also be the ratio of low frequency waves to high frequency waves as long as the format of the ratio is consistent throughout out the process. For example, the comparison may be between a ratio of high frequency waves to low frequency waves or the reverse, i.e., low frequency waves/high frequency waves.

The one or more features or values may be predetermined. For example, the range for waves that are high frequency waves may be predetermined as being greater than a set value. Similarly, the range for waves that are low frequency waves may be predetermined as being less than a set value. The set values may be the same for all patients or may vary depending on specific patient characteristics.

Other features or values of the one or more signals may be extracted. For example, signal to noise ratios may also be determined for other uses. Data quality may be assessed by looking for non-physiologic frequencies of electrical activity. Data collection and/or interpretation may be limited to stopped when data quality is below an acceptable level.

### EXAMPLES

### Example 1

### Methods and Materials

### Animals and Housing

Male wildtype C57Bl/6 mice of early adulthood (˜2-3 months) and aged mice (˜18-19 months) were purchased from Jackson Laboratory. Mice were housed at the animal housing facility at the University of Iowa. All mice were single-sex-housed in Plexiglas cages with food and water available ad libitum. Ambient temperature and relative humidity were tightly controlled in the facility. Animals were maintained in a 12:12 hour light-dark cycle within the animal facility. Animal experiments were conducted following a protocol approved by the UI Institutional Animal Care and Use Committee.

### Drugs and Reagents

LPS (Sigma, E. Coli origin, serotype O111:64, cat. #L2630) was obtained and dissolved in saline for injection. LPS and saline were injected into mice intraperitoneally.

### Experimental Design

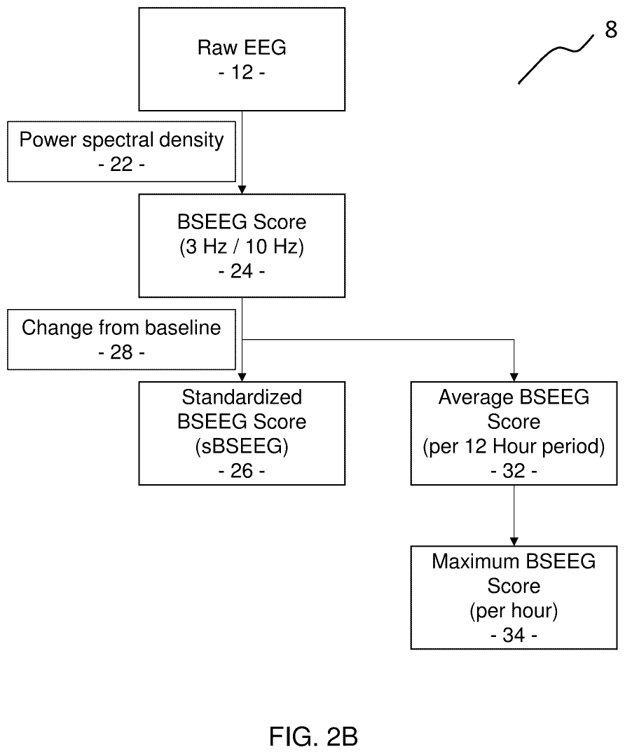

The experimental schedule of EEG head mount placement surgery, EEG recording, and inflammation experiment is shown in FIG. 3. Here on Day 1 a baseline measurement was taken. On Day 2, saline was injected and one Day 4 LPS was injected. Various dosages of LPS were injected as described herein.

### EEG Electrode Head Mount Placement Surgery

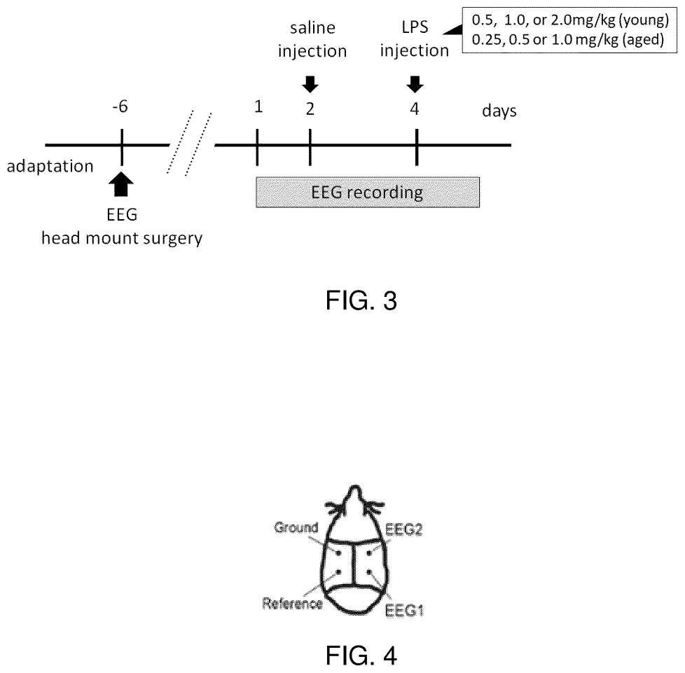

EEG electrodes were positioned on the head of mice, as shown in FIG. 4, using a standard protocol, followed by 1-week recovery from head mount surgery. The EEG recordings were stable before the start of inflammation experiment. Wired EEG systems (Sirenia EEG system; Pinnacle Technologies, Inc., Lawrence, KS) were used for the experiments following the standard procedure.

### Inflammation Experiment

The schedule was the following: Day 1, baseline measurement; Day 2, saline injection; Day 3, recovery; Day 4, LPS injection; and Days 5 and after, recovery. For each mouse, at 8 AM three cohorts of 3-4 male mice with the following doses of LPS were injected: 0.5, 1, and 2 mg/kg for young mice, and 0.25, 0.5, and 1 mg/kg for aged mice. Different dosage ranges of LPS were set between the young group and aged group because the maximum dose 2 mg/kg was expected to be fatal for aged mice. A total of 9-10 mice per each age group were included. Results were compared across groups.

### Calculation of BSEEG Score

EEG was recorded after LPS injection using wildtype early adulthood mice (˜2-3 month-old) and aged mice (˜18-19 month-old). The EEG recordings were converted for power spectral density to calculate a BSEEG score.

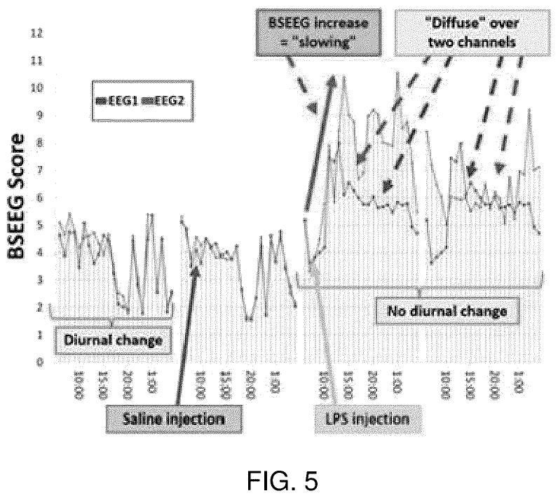

EEG Signal Processing—The algorithm was implemented to yield an EEG score to measure the presence and severity of delirium in patients. Animal EEG recordings were analyzed using a similar algorithm to that used in the prior disclosed human study. Raw EEG recordings were digitally converted to power spectral density to calculate BSEEG score(s). Specifically, SleepPro (Sirenia EEG system; Pinnacle Technologies, Inc., Lawrence, KS) software was used to export raw data after Fast Fourier Transformation, and processed it through the algorithm to calculate the BSEEG score(s). BSEEG scores are derived through a calculation of ratio between 3 Hz power to 10 Hz power. The BSEEG score was plotted on the y-axis and time of recording was plotted on the x-axis, as shown in FIG. 5.

Standardized BSEEG (sBSEEG) Score—To standardize change of the BSEEG score from baseline, the average BSEEG score during the initial 12 hours at baseline for each mouse (1st day—daytime) was set as zero, and the extent of change from that baseline was defined as standardized BSEEG (sBSEEG) score.

Maximum BSEEG Score—To compare change of BSEEG score after various doses of LPS injection, a maximum BSEEG score increase from EEG2 (frontal) after LPS injection was calculated. The BSEEG score increase was calculated as:

(BSEEG score after LPS injection every hour)−(the average BSEEG score during the initial 12 hours).

Then, the largest value was defined as maximum BSEEG score increase. The maximum BSEEG score increase was used to compare among different groups based on the dose of LPS injected.

Statistical Analyses

In order to examine the dosage effect on BSEEG score(s) and compare young mice and aged mice groups, a linear regression model including a level of dose, group, and interaction between the level and group was fitted. For estimation of coefficients in the model, generalized estimating equations using the compound symmetry working correlation structure were used to account for the within-subject correlations. Wald tests were conducted at a significance level of 0.05. All statistical analyses were performed in R.

### Web-Based BSEEG Score Calculator

A web-based tool to calculate BSEEG score from raw EEG signal data was developed and obtained through the Pinnacle Sirenia EEG system.

### Results

### Typical Pattern of BSEEG Score Changes After LPS Injection

Testing of young mice (2-3 months old, n=9) and aged mice (18-19 months old, n=10) attached to two EEG channels revealed diurnal changes over 24-hour periods, both at baseline and after injection of the LPS vehicle. FIG. 5 depicts a typical example of BSEEG scores before and after LPS injection. For simplicity, data from day 3 (one day after normal saline) was omitted. During the day while the mice remained asleep, the BSEEG score was slightly high and relatively stable. During the night when the mice were active, the BSEEG score was low on average and erratic. This diurnal change pattern was not affected after saline injection.

When the mice were injected with LPS, the BSEEG score increased indicating EEG slowing, and the score reached a peak score at around 12 hours and the diurnal changes diminished (FIG. 5). LPS injection increased BSEEG scores dose-dependently and diminished the diurnal changes. The mean BSEEG score increased more in the aged mice group as dosage increased.

### sBSEEG Score Changes After LPS Injection and Loss of Diurnal Change Over Days

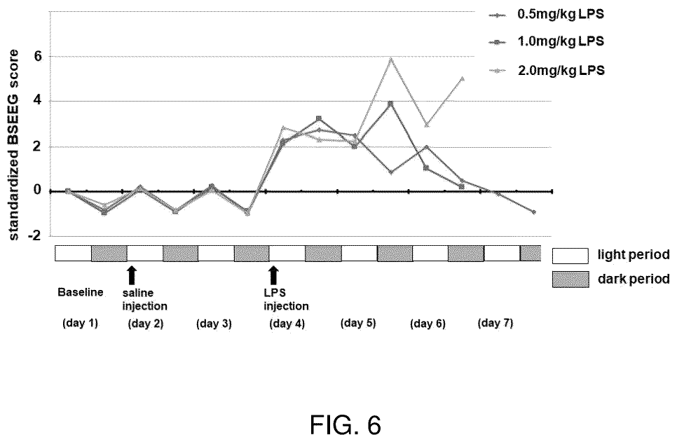

To highlight the diurnal changes, the data points in FIG. 6 represent the average sBSEEG from EEG2 (frontal) scores every 12 hours over several days. An average sBSEEG score across 3 mice in each LPS dose group over an extended period of time is shown on the y-axis. The pattern of diurnal changes for initial three days were highly consistent across multiple experiments from 9 mice tested. Even after saline injection (day 2), the pattern did not change (FIG. 6, day 1 through day 3). After LPS injection on day 4, the diurnal pattern diminishes and remains abnormal for about 3 days after LPS exposure (day 6). By day 7, diurnal changes seem to return.

### Dose-Dependent BSEEG Score Increase in Response to LPS

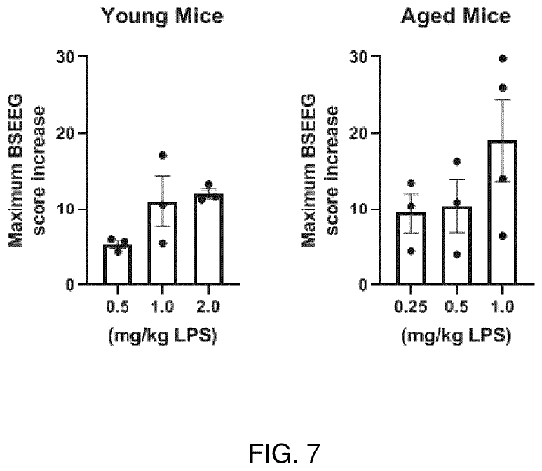

The maximum BSEEG scores from EEG2 (frontal) after various doses of LPS injection (0.5, 1, and 2 mg/kg for young mice; 0.25, 0.5, and 1 mg/kg for aged mice) were compared (FIG. 7). According to Wald tests at a significance level of 0.05, the mean score increased significantly as a level of dosage increased both in the young mice group (p-value<0.0001) and in aged mice group (p-value<0.0001). In addition, the interaction effect was statistically significant (p-value=0.004) and the estimated coefficient was positive. This indicates that the BSEEG score increased much faster in the aged group as dosage increased (FIG. 7).

### Web-Based BSEEG Score Calculator

After recording raw EEG signals, the data can be exported as an EDF file by a default software for the Pinnacle Sirenia EEG system or other appropriate file type and system. Then the EDF file is uploaded to a web-based tool, and BSEEG score can be visualized. Once sequential data from recording over days, is uploaded, sBSEEG score can be also calculated using the first day baseline data.

### Discussion

BSEEG data was used to objectively quantify brain electrophysiological changes in response to LPS injection inducing a systemic inflammation reaction in C57Bl/6 mice. This type of LPS injection animal model is widely used to evaluate consequences of systemic inflammation. Disclosed herein is the ability to quantify delirium in an animal model using EEG and in certain implementations BSEEG.

This approach is supported by the success of previously disclosed human studies, in which BSEEG was used to differentiate between patients with and without delirium. With the previously disclosed algorithm, various features of EEG raw signals were tested before developing the algorithm to distinguish patients with delirium from those without. Through this clear, quantifiable differences in the severity of delirium between patients who had experienced delirium and patients who had not were found. In the present study on a mouse model, the same algorithm was applied in analysis of EEG signals measured from a mouse.

The data of this example shows that the BSEEG method makes it possible to assess electrophysiological activities in mice similar to EEG features characteristic of delirium—consistent with the previous findings using BSEEG in humans. An increased BSEEG score indicates “EEG slow wave,” which suggests brain dysfunction. LPS injection diminished diurnal change of BSEEG score: the BSEEG score was relatively stable and slightly high during the daytime, whereas the BSEEG score was erratic and low during the night (FIGS. 3 and 4). These LPS-induced BSEEG changes are consistent with disturbances of the sleep/awake cycle in patients with delirium.

Further, aged mice reacted more strongly than young mice to LPS in a dose-dependent manner (FIG. 7). These age-associated and LPS dose-associated differences are consistent with the fact that patients with higher age or with severer systemic inflammation, such as serious infection or highly invasive surgery, are at higher risk of developing delirium.

### Example 2

Materials and Methods—In this Example C57BL/6 mice were studied, including both young (8 weeks old) and aged (72 weeks old) mice. The study also used ultrapure LPS (Invivo Gen) at various dosages ranging from 0.5-2.0 mg/kg given intraperitoneally.

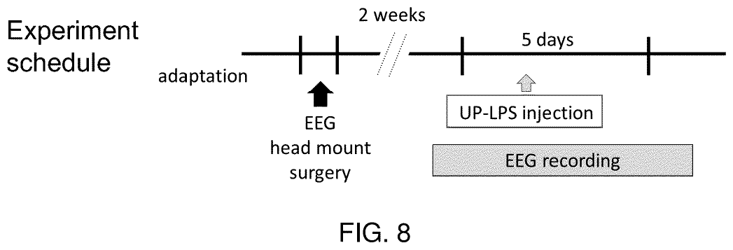

The experimental schedule is shown in FIG. 8. In the experiment the EEG head mount surgery was performed two weeks prior to EEG recording. EEG recording began on day 0 and continued for 5 days. On day 2 the mice received a UP-LPS injection.

Both wired and wireless EEG systems were used, as would be appreciated.

Analysis—EEG data was analyzed by recording raw EEG signals. This raw EEG data is then processed by an appropriate software to determine a BSEEG score (3 Hz/10 Hz). In certain instances, a web based BSEEG calculator is used.

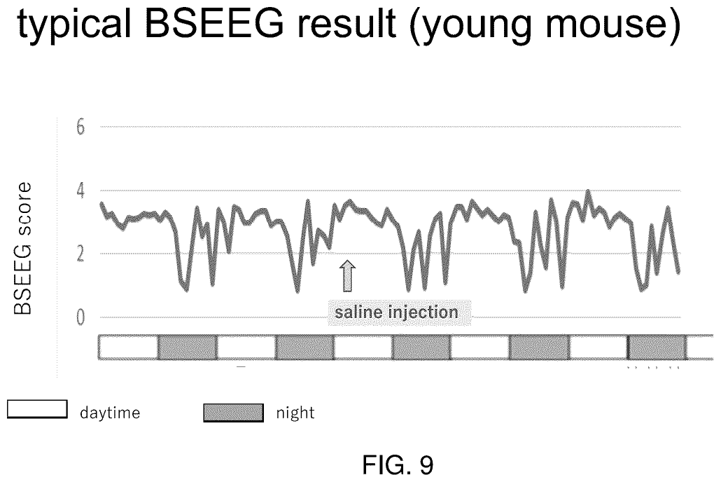

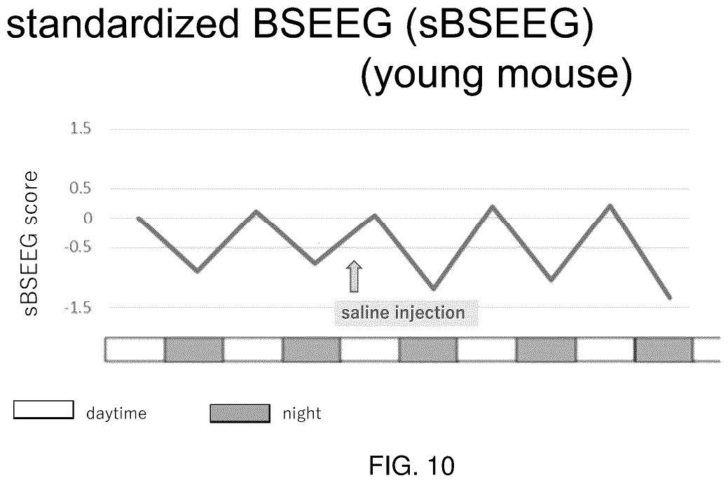

Results—FIG. 9 shows BSEEG scores for young mice over a period of days. On days 2 the mice received a saline injection. FIG. 10 shows sBSEEG scores from the data. This data shows diurnal changes in the young mice.

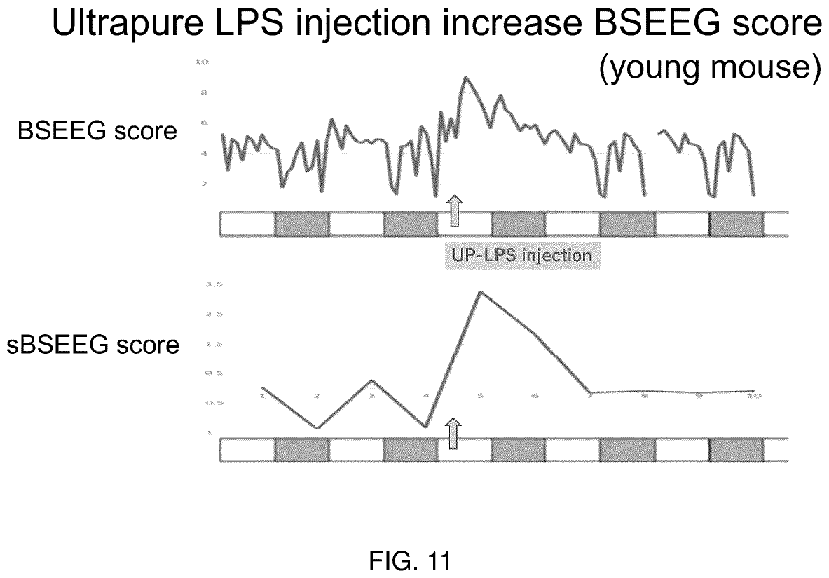

FIG. 11 shows BSEEG and sBSEEG scores for young mice where the mice received a UP-LPS injection at the indicated time. As can be seen, particularly with the sBSEEG data, the diurnal changes stopped after the LPS injection.

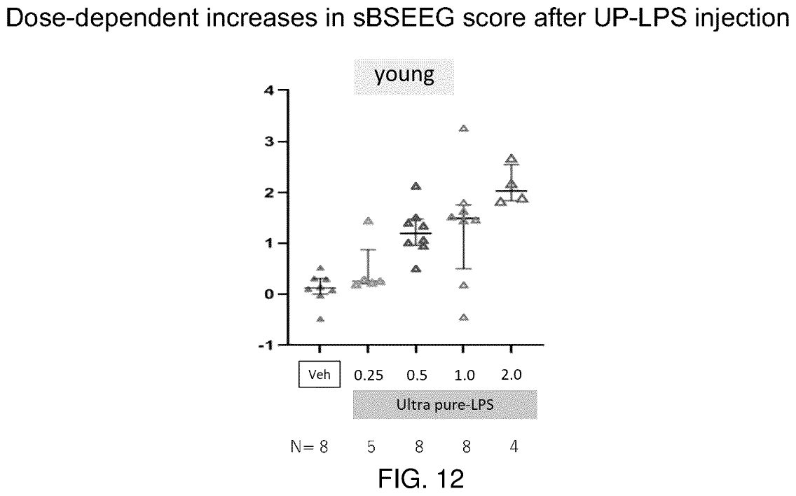

FIG. 12 shows dose-dependent increases in sBSEEG scores after UP-LPS injection in young mice. That is as the dosage of UP-LPS increased so to did the sBSEEG score after administration.

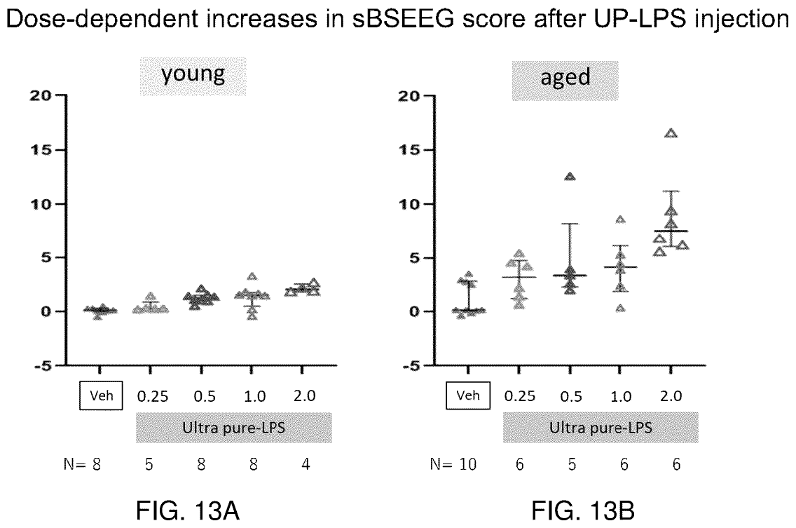

FIGS. 13A-B show the dose-dependent changes in sBSEEG scores after UP-LPS injection in both young (FIG. 13A) and agreed (FIG. 13B). As can be seen in both young and aged mice there is an increase in sBSEEG scores when the dose of UP-LPS administered also increased. The rate of increase was greater in aged mice compared to young mice.

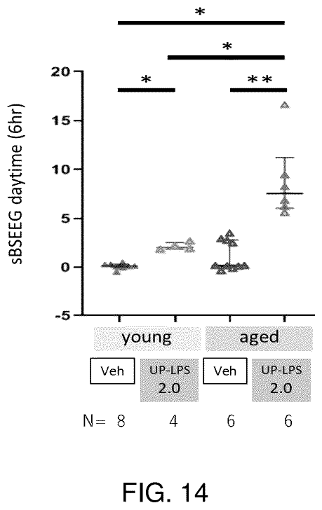

FIG. 14 shows that with a 2.0 mg/kg dose of UP-LPS in both young and aged mice there is an increase in the BSEEG score. The UP-LPS induced a larger BSEEG score increase in aged mice compared to the young mice.

Although the disclosure has been described with references to various embodiments, persons skilled in the art will recognized that changes may be made in form and detail without departing from the spirit and scope of this disclosure.

What is claimed is:

1. A bispecteral EEG assessment system, comprising:
a) at least one sensor;
b) a screening device; and
c) a platform tool, the system being configured to:
record an EEG to obtain raw EEG data;
calculate a recorded BSEEG score; and
compare the recorded BSEEG score to a clinical inflammation threshold.

2. The system of claim 1, wherein the raw EEG data is recorded over a time period.

3. The system of claim 1, further comprising calculating a maximum BSEEG score.

4. The system of claim 1, further comprising outputting the recorded BSEEG score.

5. The system of claim 1, wherein the system is configured to update the clinical inflammation threshold via a machine learning model.

6. The system of claim 1, wherein the platform tool is a web-based tool.

7. The system of claim 1, further configured to quantify the level of brain wave abnormality and the level of neuroinflammation over time.

8. The system of claim 1, wherein the recorded BSEEG score is calculated from raw EEG data via power spectral density analysis.

9. The system of claim 8, where the recorded BSEEG score is a ratio of EEG signals at 3 Hz to 10 Hz.

10. The system of claim 1, further comprising calculating a baseline BSEEG score.

11. The system of claim 10, further comprising calculating a standardized BSEEG score.

12. A system for quantifying neuroinflammation, comprising:
(a) at least two sensors configured to record EEG signals indicative of brain wave frequencies;
(b) a processor; and
(c) at least one tool configured to run on the processor, the tool configured to:
record EEG signals;
perform spectral density analysis on the EEG signal to calculate a BSEEG score; and
compare the BSEEG score to a clinical inflammation threshold to determine an amount of neuroinflammation.

13. The system of claim 12, wherein the at least one tool is configured to determine a baseline BSEEG score.

14. The system of claim 12, wherein the at least one tool is configured to calculate a standardized BSEEG score.

15. The system of claim 12, wherein the at least one tool is configured to calculate a maximum BSEEG score over a given time period.

16. The system of claim 12, wherein the at least one tool is configured to calculate an average BSEEG score over a given time period.

17. The system of claim 12, further comprising a display configured to display output including an average BSEEG score, a maximum BSEEG score, a baseline BSEEG score, a standardized BSEEG score.

18. The system of claim 12, wherein the tool is further configured to quantify a level of brain wave abnormality and a level of neuroinflammation over time.

19. The system of claim 12, wherein the given time period is about 12 hours.

20. An inflammation assessment method, comprising:
recording an EEG to obtain raw EEG data;
calculating a recorded BSEEG score; and
comparing the recorded BSEEG score to a clinical inflammation threshold.
